(19) United States
(12) Patent Application Publication
(10) Pub. No.: US 2024/0189119 A1
(43) Pub. Date: Jun. 13, 2024

(54) ARTIFICIAL MUSCLE ACTUATOR DEVICE

(71) Applicant: MOUMUS CONSULTING GROUP LLC, Suzaka-shi, Nagano (JP)

(72) Inventor: Shouzou MIYAZAWA, Suzaka-shi, Nagano (JP)

(21) Appl. No.: 18/286,823

(22) PCT Filed: Apr. 26, 2022
(86) PCT No.: PCT/JP2022/018825

§ 371 Date: Oct. 13, 2023

(30) Foreign Application Priority Data
May 14, 2021 (JP) 2021-082110
Aug. 30, 2021 (JP) 2021-139817
Apr. 13, 2022 (JP) 2022-065968

(51) Int. Cl. A61F 2/74; B25J 19/00; F15B 15/10

(52) U.S. Cl. CPC A61F 2/741; B25J 19/00; F15B 15/103

(57) ABSTRACT

The artificial muscle actuator device is structured to suit the muscles of the body and has a versatile structure that can be applied as a device responsible for the motion function of a robot, due to a small, electric-power-conserving motive power source that uses balloon inflation and deflation and eliminates fluid pressure loss. In the artificial muscle actuator device, an actuator unit and a first artificial muscle unit are connected by a first linking tube, the actuator unit and a second artificial muscle unit are connected by a second linking tube, the first artificial muscle unit and the second artificial muscle unit are attached to a body site, and the actuator unit is controlled by current conduction, so as to perform flexion and extension movements of a skeleton at the body site.

### TECHNICAL FIELD

The present invention relates to an artificial muscle actuator device.

### BACKGROUND ART

An artificial muscle actuator device that assists a motion function of a body is known, and attempts to approximate the motion of the body by using a tube for an artificial muscle have been made (PTL 1: JP-A-2015-157346, PTL 2: Japanese Patent No. 5246717).

### CITATION LIST

### Patent Literature

PTL 1: JP-A-2015-157346
PTL 2: Japanese Patent No. 5246717

### SUMMARY OF INVENTION

### Technical Problem

The prior art uses a large-sized compressor, and consumes power too much. In addition, the prior art lacks compatibility with the body because it has a mechanical shape different from the muscles of the body. The prior art has a problem that it is conceivable to more approximate the shape thereof to the shape of the muscles of the body, but the versatility is poor, for example, it is not possible to apply the prior art to a device that handles the motion function of a robot.

### Solution to Problem

The present invention has been made in view of the above-described circumstances, and an object of the present invention is to provide an artificial muscle actuator device having a structure that is suitable for muscles of a body by using a compact and power-saving power source in which inflation and deflation of a balloon make a pair and a loss of working fluid pressure is eliminated, and the device having a structure that can be applied as a device that handles a motion function of a robot and is excellent in versatility.

The present invention has been accomplished under the solutions as disclosed below.

According to the present invention, an artificial muscle actuator device includes a first artificial muscle unit, a second artificial muscle unit, and an actuator unit. The artificial muscle actuator device is configured to be attached to a body part corresponding to a skeleton in which a first bone and a second bone are linked by a joint, and to perform a bending motion and a stretching motion with the first artificial muscle unit and the second artificial muscle unit. The first artificial muscle unit includes a first balloon that is inflated and deflated by fluid pressure of a first working fluid, a first joining tube that joins the first balloon and the actuator unit to each other, and a first net that covers an outer periphery of the first balloon and is attached to the body part. The second artificial muscle unit includes a second balloon that is deflated and inflated by fluid pressure of a second working fluid, a second joining tube that joins the second balloon and the actuator unit to each other, and a second net that covers an outer periphery of the second balloon and is attached to the body part. The actuator unit includes a housing to which each of the first joining tube and the second joining tube is joined, a piston disposed in the housing, and a solenoid that moves the piston back and forth, and is configured to perform a first motion of pushing the first working fluid toward the first balloon and drawing the second working fluid from the second balloon via a forward movement of the piston, and a second motion of pushing the second working fluid toward the second balloon and drawing the first working fluid from the first balloon via a backward movement of the piston.

According to this configuration, since it is possible to make a structure suitable for muscles of a body, it is possible to assist a motion function of the body. In addition, it is possible to make a structure that can be applied as a device that handles a motion function of a robot and is excellent in versatility. Further, since the actuator unit is configured to increase and decrease working fluid pressure, it is possible to make an energy-saving structure in which a loss of the working fluid pressure of the balloon is eliminated. In addition, it is possible to reduce the size, and to make a rational configuration with a small number of components.

According to the present invention, an artificial muscle actuator device includes a first artificial muscle unit, a second artificial muscle unit, and an actuator unit. The artificial muscle actuator device is configured to be attached to a body part corresponding to a skeleton in which a first bone and a second bone are linked by a joint, and to perform a bending motion and a stretching motion with the first artificial muscle unit and the second artificial muscle unit. The first artificial muscle unit includes a first balloon that is inflated and deflated by fluid pressure of a first working fluid, a first joining tube that joins the first balloon and the actuator unit to each other, and a first net that covers an outer periphery of the first balloon and is attached to the body part. The second artificial muscle unit includes a second balloon that is deflated and inflated by fluid pressure of a second working fluid, a second joining tube that joins the second balloon and the actuator unit to each other, and a second net that covers an outer periphery of the second balloon and is attached to the body part. The actuator unit includes a housing to which each of the first joining tube and the second joining tube is joined, a first piston disposed in the housing, a first solenoid that moves the first piston back and forth, a second piston disposed in the housing, and a second solenoid that moves the second piston back and forth, and is configured to perform a first motion of pushing the first working fluid toward the first balloon and drawing the second working fluid from the second balloon via a motion of separating the first piston and the second piston away from each other, and a second motion of pushing the second working fluid toward the second balloon and drawing the first working fluid from the first balloon via a motion of bringing the first piston and the second piston close to each other.

According to this configuration, since it is possible to make a structure suitable for muscles of a body, it is possible to assist a motion function of the body. In addition, it is possible to make a structure that can be applied as a device that handles a motion function of a robot and is excellent in versatility. Further, since the actuator unit is configured to increase and decrease working fluid pressure, it is possible to make an energy-saving structure in which a loss of the working fluid pressure of the balloon is eliminated. In addition, it is possible to increase the power and to raise and lower the fluid pressure in a short time, so that it is possible to more increase a motion speed.

As an example, the first artificial muscle unit is configured such that a first end portion of the first artificial muscle unit at a position close to the first joining tube is joined to a position on a bending side of the second bone at a position far from the joint in the first bone, and a second end portion at a position far from the first joining tube is joined to a position on the bending side at a position close to the joint in the first bone. The second artificial muscle unit is configured such that a third end portion of the second artificial muscle unit at a position close to the second joining tube is joined to a position on a stretching side of the second bone at the position far from the joint in the first bone, and a fourth end portion at a position far from the second joining tube is joined to a position on the stretching side at the position close to the joint in the first bone. With this configuration, it is possible to attach the device to the optimum position of the body part and to make it easy to efficiently operate the device.

Preferably, a configuration of further including a first attachment tool that attaches, to the body part, a first connection portion of the first joining tube with a first end portion of the first net on a rear end side of the first net, and a second connection portion of the second joining tube with a third end portion of the second net on a rear end side of the second net, and a second attachment tool that attaches, to the body part, a second end portion of the first net on a front end side of the first net and a fourth end portion of the second net on a front end side of the second net is made. With this configuration, it is possible to be easily attached to and detached from the body part, and to make it easy to be aligned with the optimum position of the body part. As an example, the first attachment tool and the second attachment tool are configured to include a cylindrical portion for attaching a balloon end portion and a net end portion, an L-shaped portion attached to the body part, and a rib portion for reinforcing the cylindrical portion and the L-shaped portion. With this configuration, it is possible to make it easy to assemble the artificial muscle unit and to attach the artificial muscle unit to the body part.

Preferably, both the first balloon and the second balloon are made of oil-resistant elastic rubber, both the first net and the second net are made of synthetic-fiber cylindrical fabric, the cylindrical fabric has a plurality of thread-like fibers each being pulled out from both end portions, and the thread-like fibers are moveable with the cylindrical fabric. With this configuration, it is possible to extend a life of the motion of repeating inflation and deflation to be long. As a more preferable configuration, the first balloon and the second balloon are made of silicone rubber, fluororubber, urethane rubber, or natural rubber, and the first net and the second net are made of carbon fiber, polyester fiber, or polyamide fiber. With this configuration, it is possible to make a structure having high strength and high flexibility, and to extend the life of the motion of repeating inflation and deflation to be long.

As an example, the first working fluid and the second working fluid are flame-retardant working oils. As a result, it is possible to make a highly safe structure. As an example, the housing is configured to be provided with an adjustment valve for adjusting the fluid pressure of the first working fluid. Thus, it is possible to make it easy to perform balance adjustment between the fluid pressure of the first working fluid and the fluid pressure of the second working fluid. In other words, although known actuators using working oils have a bottleneck due to changes in operating pressure due to the temperature and atmospheric pressure, it is possible to make it easy to keep the desired driving pressure constant by providing the adjustment valve.

As an example, both the first net and the second net are plain weave and are cylindrical. As an example, both the first net and the second net are made of tough fibers such as carbon fiber, and thus it is possible to perform an inflation motion and a deflation motion similar to motions of muscles of the body while protecting the first balloon and the second balloon from external forces. As an example, since it is possible to make all members of the artificial muscle unit with organic materials, it is possible to obtain excellent affinity with the muscles of the body and to reduce an environmental load.

The skeleton may be a skeleton of a human or the like, or a skeleton of a robot. The joining structure may be directly fixed, or may be indirectly fixed via an interposition such as clothing or a belt. Therefore, the artificial muscle actuator device according to the present invention can assist the motion function of the body. Also, the artificial muscle actuator device according to the present invention can be applied to a device that handles a motion function of a robot.

As an example, preferably, the actuator unit includes a housing in which a first connection port is joined to the first joining tube, and a second connection port is joined to the second joining tube, and an adjustment valve for adjusting fluid pressure of the first working fluid is disposed, a first solenoid that moves a first piston back and forth in the housing, and a second solenoid that moves a second piston back and forth in the housing. Preferably, when the actuator unit performs the first motion, either or both of a configuration in which the first piston pressurizes the first working fluid and depressurizes the second working fluid, and a configuration in which the second piston pressurizes the first working fluid and depressurizes the second working fluid are made. Preferably, when the actuator unit performs the second motion, either or both of a configuration in which the first piston depressurizes the first working fluid and pressurizes the second working fluid, and a configuration in which the second piston depressurizes the first working fluid and pressurizes the second working fluid are made. According to this configuration, it is possible to inflate or deflate one of a pair of balloons by whether any or all of a plurality of solenoids are energized, or a magnetic pole change operation in an energization direction, and to freely operate a bending/stretching angle of the skeleton or an artificial skeleton by displacement of the entire length of both end portions of the cylindrical plain woven net.

As an example, the actuator unit is a solenoid in which a piston as a permanent magnet moves back and forth. As an example, the actuator unit is configured to include a linear solenoid in which a piston that is a permanent magnet or a piston in which electromagnetic soft iron is disposed moves back and forth in a state of being surrounded by a solenoid coil. According to this configuration, it is possible to make it easy to feed the working fluid with high efficiency. By adopting a neodymium magnet as the permanent magnet, it is possible to make a particularly high output.

According to the present invention, it is possible to assist a motion function of a body with a structure suitable for muscles of the body by using a compact and power-saving power source in which inflation and deflation of a balloon make a pair and a loss of working fluid pressure is eliminated. In addition, it is possible to realize an artificial muscle actuator device having a structure that can be applied as a device that handles a motion function of a robot and is excellent in versatility.

### BRIEF DESCRIPTION OF DRAWINGS

FIG. 1 is a schematic structural diagram schematically illustrating a state in which an artificial muscle actuator device in a first example according to an embodiment of the present invention is attached to a body part (skeleton) and a bending motion is performed.

FIG. 2 is a schematic structural diagram schematically illustrating a state in which the artificial muscle actuator device in the first example is attached to the body part (skeleton) and a stretching motion is performed.

FIG. 3A is a schematic structural diagram schematically illustrating a state in which the artificial muscle actuator device in the first example is attached to the body part and an A motion of walking is performed, and FIG. 3B is a schematic structural diagram schematically illustrating a state in which the artificial muscle actuator device in the first example is attached to the body part and a B motion of walking is performed.

FIG. 4A is a schematic structural diagram schematically illustrating a state in which the artificial muscle actuator device in the first example is attached to the body part and a standing motion is performed, and FIG. 4B is a schematic structural diagram schematically illustrating a state in which the artificial muscle actuator device in the first example is attached to the body part and a knee-bending motion is performed.

FIG. 5 is a schematic structural diagram schematically illustrating a state in which an artificial muscle actuator device in a second example according to the embodiment of the present invention is attached to a body part (skeleton) and a bending motion is performed.

FIG. 6 is a schematic structural diagram schematically illustrating a state in which the artificial muscle actuator device in the second example is attached to the body part (skeleton) and a stretching motion is performed.

FIG. 7A is a side view illustrating an inflated state of a balloon according to the present embodiment, FIG. 7B is a side view illustrating inflated states of the balloon and a net according to the present embodiment, FIG. 7C is a side view illustrating the inflated states of the balloon and the net according to the present embodiment from another direction, FIG. 7D is a side view illustrating a deflated state of the balloon in FIG. 7A, and FIG. 7E is a side view illustrating deflated states of the balloon and the net in FIG. 7B.

FIG. 8A is a side view illustrating an example of an attachment plate according to the present embodiment, FIG. 8B is a plan view of FIG. 8A, and FIG. 8C is a front view of FIG. 8A.

### DESCRIPTION OF EMBODIMENTS

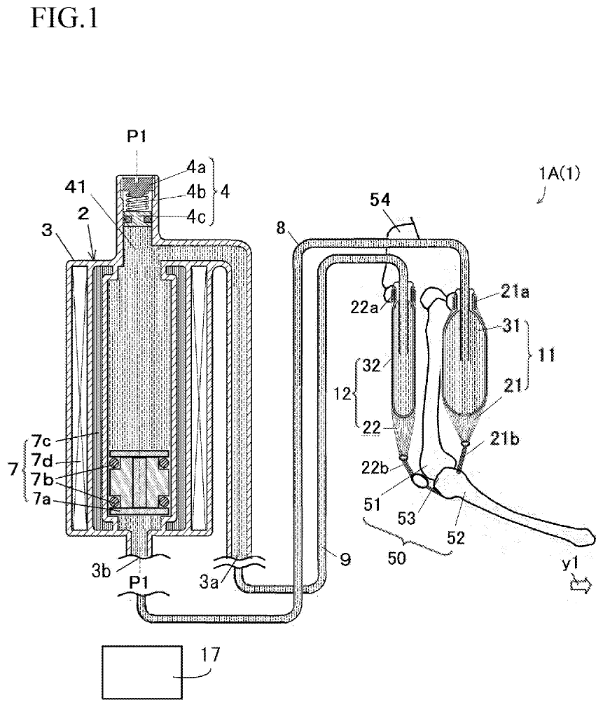

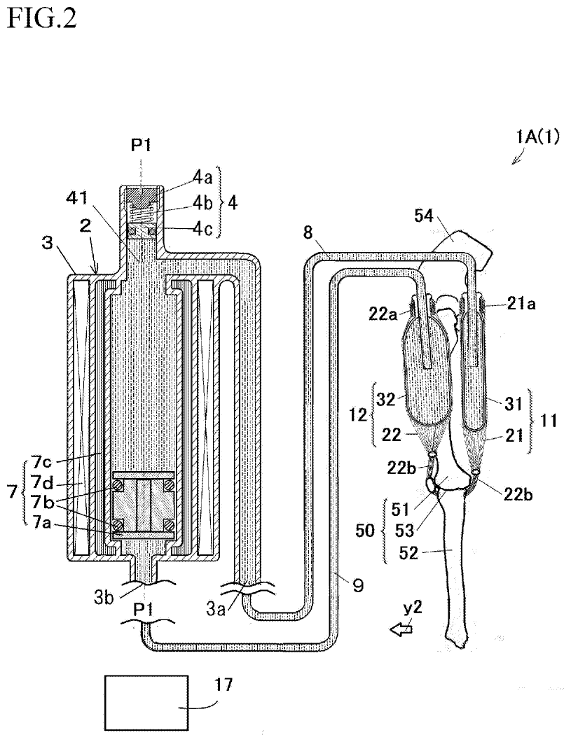

Hereinafter, an embodiment according to the present invention will be described in detail with reference to the drawings. An artificial muscle actuator device 1 according to the present embodiment is used in a manner that an actuator unit 2 and a first artificial muscle unit 11 are connected by a first joining tube 8, the actuator unit 2 and a second artificial muscle unit 12 are connected by a second joining tube 9, and the first artificial muscle unit 11 and the second artificial muscle unit 12 are attached to a body part 54 corresponding to a skeleton 50 in which a first bone 51 and a second bone 52 are linked by a joint 53. A configuration in which a control circuit 17 controls energization of the actuator unit 2 to perform a bending motion and a stretching motion of the skeleton 50 is made. Here, the bending motion is a state where the position of the second bone 52 far from the joint 53 moves in a y1 direction, as illustrated in FIG. 1, for example. Here, the stretching motion is a state where the position of the second bone 52 far from the joint 53 moves in a y2 direction (the opposite direction of the y1 direction), as illustrated in FIG. 2, for example. In all drawings for describing the embodiment, the same reference numerals will be assigned to members having the same function, and repeated description thereof may be omitted in some cases.

The present embodiment is configured to include a first balloon 31 that is inflated and deflated by the fluid pressure of a first working fluid 41 and a first net 21 that covers the outer periphery of the first balloon 31 and is attached to the body part 54. In addition, a configuration in which a second balloon 32 that is inflated and deflated by the fluid pressure of a second working fluid 42 and a second net 22 that covers the outer periphery of the second balloon 32 and is attached to the body part 54 is made. Here, the first working fluid 41 and the second working fluid 42 are flame-retardant working oils.

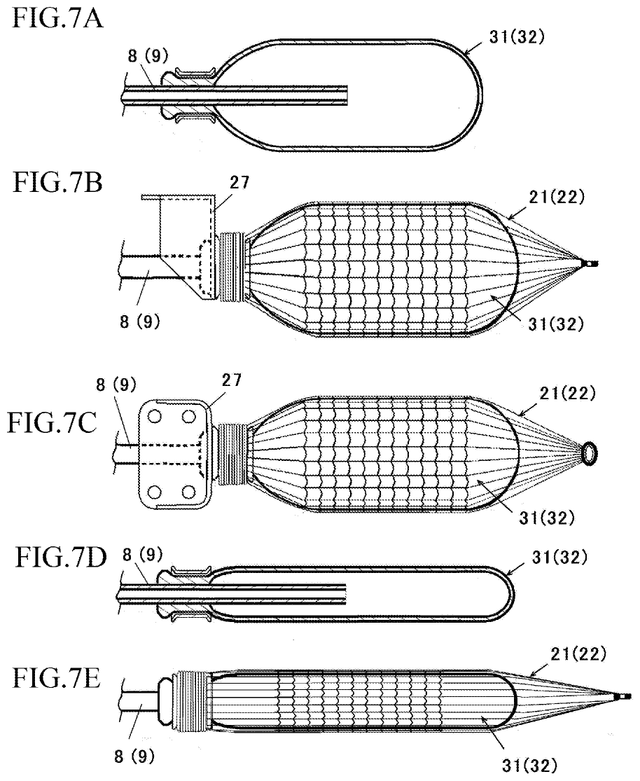

As illustrated in FIGS. 7A and 7D, as an example, oil-resistant elastic rubber such as silicone rubber is formed into a columnar shape to form the first balloon 31 and the second balloon 32 having a structure that can be easily inflated and deflated. Further, as illustrated in FIGS. 7B and 7E, as an example, a configuration in which the first net 21 and the second net 22 are made of cylindrical fabric of synthetic fiber such as carbon fiber, the cylindrical fabric has a plurality of thread-like fibers each being pulled out from the entire circumference of both end portions at equal intervals, and the thread-like fiber is moveable with the cylindrical fabric is made. Then, the first balloon 31 linked to the first joining tube 8 is covered with the first net 21 to make a structure that facilitates inflation and deflation together with the first balloon 31. Similarly, the second balloon 32 linked to the second joining tube 9 is covered with the second net 22 to make a structure that facilitates inflation and deflation together with the second balloon 32. Then, the first balloon 31 is filled with the first working fluid 41 to make the first artificial muscle unit 11. Similarly, the second balloon 32 is filled with the second working fluid 42 to make the second artificial muscle unit 12.

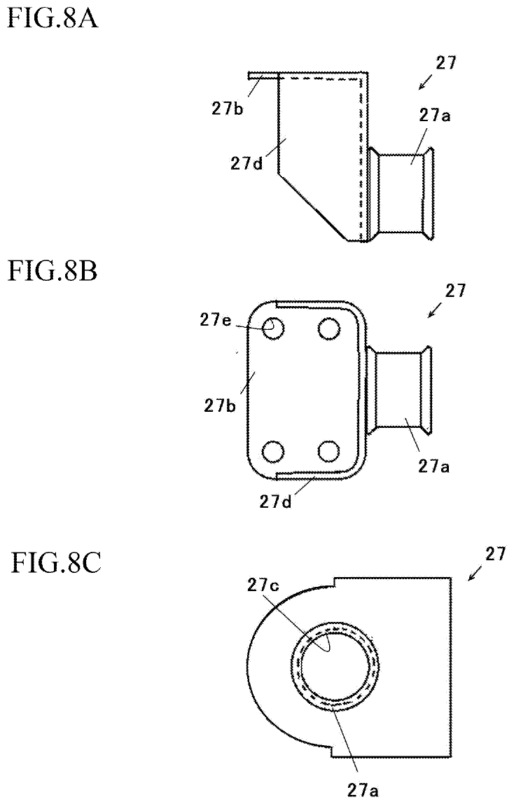

As an example, as illustrated in FIGS. 7B, 7C, and 8A to 8C, the first artificial muscle unit 11 and the second artificial muscle unit 12 may be configured to be provided with an attachment plate 27 including a cylindrical portion 27a, an L-shaped portion 27b, and a rib portion 27d. As an example, the attachment plate 27 fixes a first end portion 21a of the first balloon 31 in the first artificial muscle unit 11 through a through-hole 27c formed in the cylindrical portion 27a with an adhesive or the like. Also, one end portion of the first net 21 in the first artificial muscle unit 11 is wrapped around the outer periphery of the cylindrical portion 27a and fixed with an adhesive or the like. The L-shaped portion 27b of the attachment plate 27 is aligned with the body part 54, and bolts, screws, and the like are fixed to the body part 54 by being passed through an attachment hole 27e formed in the L-shaped portion 27b. Alternatively, the L-shaped portion 27b is fixed via a first attachment tool 23 which will be described later. The same applies to the second artificial muscle unit 12.

Next, an artificial muscle actuator device 1A in a first example will be described below.

### First Example

As illustrated in FIGS. 1 and 2, in the actuator unit 2, a housing 3, a solenoid 7 that moves a piston 7a back and forth in the housing 3, an adjustment valve 4, and the piston 7a are disposed in series along an axis P1.

In the solenoid 7, a cylindrical yoke 7c made of electromagnetic soft steel is disposed on an outside of an outer peripheral position of the piston 7a, and an electromagnetic coil 7d is disposed in an outer periphery of the yoke 7c. A moving direction of the piston 7a is determined by a direction of a current applied to the electromagnetic coil 7d. Further, a pushing force and an attracting force of the piston 7a are determined by the magnitude of electric power applied to the electromagnetic coil 7d. The piston 7a is electromagnetic soft steel or a neodymium magnet formed in a cylindrical shape. An O-ring 7b is disposed in a recess portion on the outer peripheral side surface of the piston 7a and is slid on the axis P1 along an inner wall of the housing 3 provided at a position of the inside of the yoke 7c.

The adjustment valve 4 has a configuration in which a screw 4a, a coil spring 4b, and a plunger 4c are joined along the axis P1 and is adjusted in a direction of increasing the fluid pressure in the stationary state of the first working fluid 41 by increasing the pushing amount of the screw 4a.

As illustrated in FIGS. 1 and 2, the first artificial muscle unit 11 includes the first balloon 31 that is inflated and deflated by the fluid pressure of the first working fluid 41 and the first net 21 that covers the outer periphery of the first balloon 31. In addition, the second artificial muscle unit 12 includes the second balloon 32 that is inflated and deflated by the fluid pressure of the second working fluid 42 and the second net 22 that covers the outer periphery of the second balloon 32. Both the first net 21 and the second net 22 are plain weave and cylindrical. Both the first net 21 and the second net 22 are made of tough fibers such as carbon fibers. Both the first balloon 31 and the second balloon 32 are bags made of rubber such as silicone rubber. The first joining tube 8 and the second joining tube 9 are pressure-resistant tubes. Both the first working fluid 41 and the second working fluid 42 are flame-retardant working oils.

The first end portion 21a of the first artificial muscle unit 11 at a position close to the first joining tube 8 is joined to a position on the bending side of the second bone 52 at a position far from the joint 53 in the first bone 51. In addition, a second end portion 21b of the first artificial muscle unit 11 at a position far from the first joining tube 8 is joined to a position on the bending side of the second bone 52 at a position close to the joint 53 in the first bone 51. A third end portion 22a of the second artificial muscle unit 12 at a position close to the second joining tube 9 is joined to a position on the stretching side of the second bone 52 at a position far from the joint 53 of the first bone 51. In addition, a fourth end portion 22b of the second artificial muscle unit 12 at a position far from the second joining tube 9 is joined to a position on the stretching side of the second bone 52 at a position close to the joint 53 in the first bone 51.

With the forward movement of the piston 7a, the first working fluid 41 in the housing 3 is moved into the first balloon 31 via a first connection port 3a and the first joining tube 8 by the pushing force, and thus the first balloon 31 is inflated. In addition, with the forward movement of the piston 7a, the second working fluid 42 in the second balloon 32 is moved into the housing 3 via the second joining tube 9 and a second connection port 3b by the attracting force, and thus the second balloon 32 is deflated.

Furthermore, with the backward movement of the piston 7a, the first working fluid 41 in the first balloon 31 is moved into the housing 3 via the first joining tube 8 and the first connection port 3a by the attracting force, and thus the first balloon 31 is deflated. In addition, with the backward movement of the piston 7a, the second working fluid 42 in the housing 3 is moved into the second balloon 32 via the second connection port 3b and the second joining tube 9 by the pushing force, and thus the second balloon 32 is inflated.

Here, the control circuit 17 drives and controls the actuator unit 2 by controlling the magnitude of the electric power applied to the actuator unit 2 and the direction of the energizing current.

The skeleton 50 is not limited to the skeleton of a person, and may be applied to the skeleton of a robot or the like. A joining structure between the first artificial muscle unit 11 and the second artificial muscle unit 12, and the skeleton 50 may be directly fixed, or may be indirectly fixed via an interposition such as clothing or a belt. Therefore, the artificial muscle actuator device 1 according to the present embodiment can assist the motion function of the body. Also, the artificial muscle actuator device 1 according to the present embodiment can be applied to a device that handles a motion function of a robot.

Next, a configuration for power-assisting the motions of the quadriceps and hamstrings will be described below.

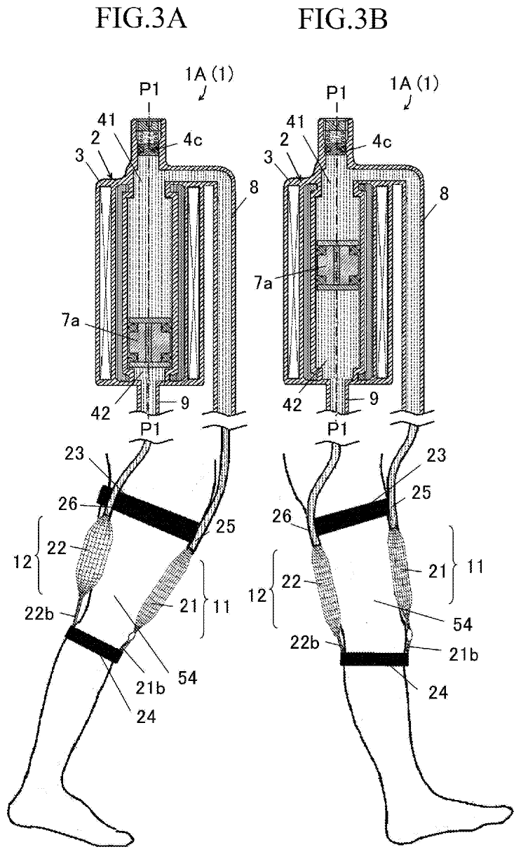

FIG. 3A is a schematic structural diagram schematically illustrating a state in which the artificial muscle actuator device in the first example is attached to the body part and an A motion of walking is performed. FIG. 3B is a schematic structural diagram schematically illustrating a state in which the artificial muscle actuator device in the first example is attached to the body part and a B motion of walking is performed.

As an example, the artificial muscle actuator device includes the band-like first attachment tool 23 for attaching, to the body part 54, a first connection portion 25 of the first joining tube 8 with the first end portion 21a of the first net 21 on the rear end side of the first net 21 and a second connection portion 26 of the second joining tube 9 with the third end portion 22a of the second net 22 on the rear end side of the second net 22. Also, the artificial muscle actuator device further includes a band-like second attachment tool 24 for attaching the second end portion 21b of the first net 21 on the front end side of the first net 21 and the fourth end portion 22b of the second net 22 on the front end side of the second net 22 to the body part 54. The first attachment tool 23 and the second attachment tool 24 are band-like members made of an elastic band or narrow fabric, and are wound around the body part and fixed in position. Alternatively, the first attachment tool 23 and the second attachment tool 24 are wound around the body part via clothes and fixed in position. The first attachment tool 23 and the second attachment tool 24 are spaced apart from each other with the joint 53 interposed therebetween, and are attached at positions where an influence on the body is relatively small, with a winding force sufficient to prevent positional deviation.

In the example illustrated in FIG. 3A, with the backward movement of the piston 7a, the first working fluid 41 in the first balloon 31 is moved into the housing 3 by the attracting force, and thus the first balloon 31 is deflated. In addition, with the backward movement of the piston 7a, the second working fluid 42 in the housing 3 is moved into the second balloon 32 by the pushing force, and thus the second balloon 32 is inflated. In this manner, the legs are moved backward and the body is moved forward. In the example illustrated in FIG. 3B, with a restoring force when the piston 7a is brought back to the origin position, the legs are moved forward and the body is brought back to the stationary position.

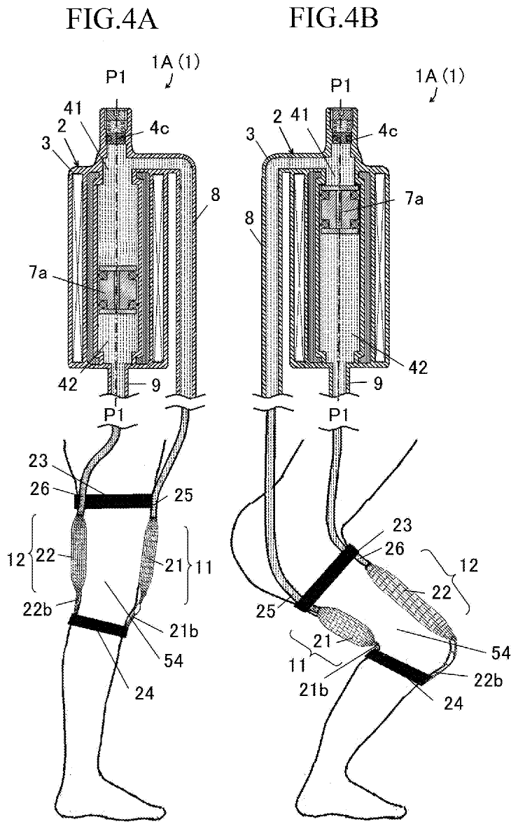

FIG. 4A is a schematic structural diagram schematically illustrating a state in which the artificial muscle actuator device in the first example is attached to the body part and a standing motion is performed. FIG. 4B is a schematic structural diagram schematically illustrating a state in which the artificial muscle actuator device in the first example is attached to the body part and a knee-bending motion is performed.

In the example illustrated in FIG. 4A, the piston 7a is slightly double-moved from the origin position to bring the body into a standing posture. In the example illustrated in FIG. 4B, with the forward movement of the piston 7a, the first working fluid 41 in the housing 3 is moved into the first balloon 31 by the pushing force, and thus the first balloon 31 is inflated. In addition, with the forward movement of the piston 7a, the second working fluid 42 in the second balloon 32 is moved into the housing 3 by the attracting force, and thus the second balloon 32 is deflated. In this manner, a knee-bending motion is performed. The above-described motion is an example, and it is possible to make it easy to perform various body motions such as a walking motion and a bending and stretching motion by driving and controlling the actuator unit 2.

Next, an artificial muscle actuator device 1B in a second example will be described below.

### Second Example

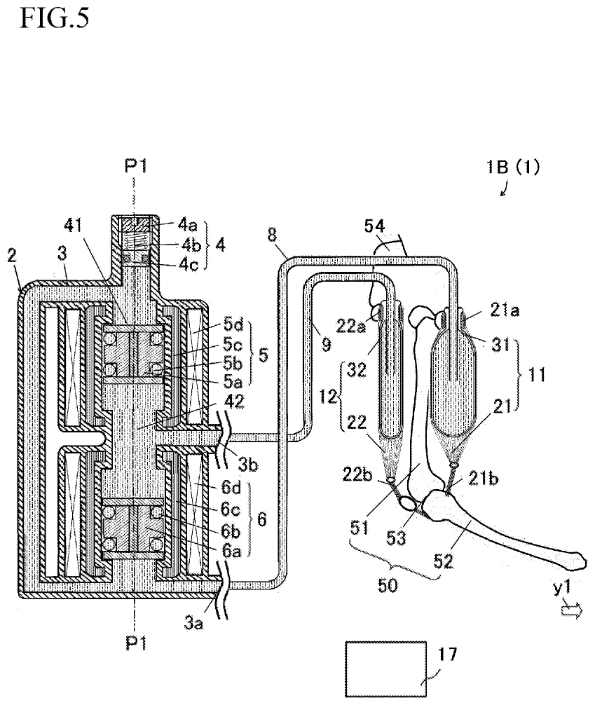

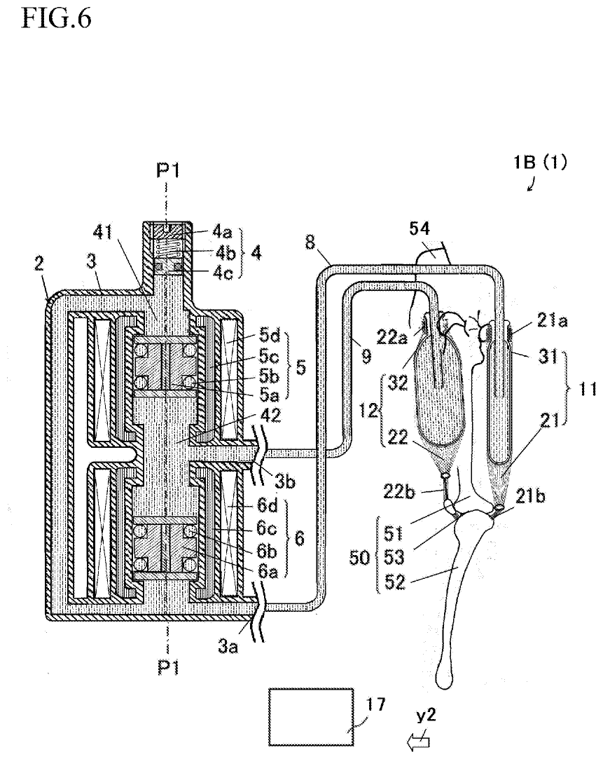

As illustrated in FIGS. 5 and 6, the actuator unit 2 includes the housing 3, a first solenoid 5 that moves a first piston 5a back and forth in the housing 3, a second solenoid 6 that moves a second piston 6a back and forth in the housing 3, and the adjustment valve 4. The first piston 5a and the second piston 6a are disposed in series along the axis P1. The adjustment valve 4 and the first piston 5a are disposed in series along the axis P1.

In the first solenoid 5, a cylindrical first yoke 5c made of electromagnetic soft steel is disposed on an outside of an outer peripheral position of the first piston 5a, and a first electromagnetic coil 5d is disposed in an outer periphery of the first yoke 5c. A moving direction of the first piston 5a is determined by a direction of a current applied to the first electromagnetic coil 5d. Further, a pushing force and an attracting force of the first piston 5a are determined by the magnitude of electric power applied to the first electromagnetic coil 5d. The first piston 5a is electromagnetic soft steel or a neodymium magnet formed in a cylindrical shape. A plurality of first O-rings 5b are disposed in a recess portion on the outer peripheral side surface of the first piston 5a and the first piston 5a is slid on the axis P1 along an inner wall of the housing 3 provided at a position of the inside of the first yoke 5c.

In the second solenoid 6, a cylindrical second yoke 6c made of electromagnetic soft steel is disposed on an outside of an outer peripheral position of the second piston 6a, and a second electromagnetic coil 6d is disposed in an outer periphery of the second yoke 6c. A moving direction of the second piston 6a is determined by a direction of a current applied to the second electromagnetic coil 6d. Further, a pushing force and an attracting force of the second piston 6a are determined by the magnitude of electric power applied to the second electromagnetic coil 6d. The second piston 6a is electromagnetic soft steel or a neodymium magnet formed in a cylindrical shape. A plurality of second O-rings 6b are disposed in a recess portion on the outer peripheral side surface of the second piston 6a and the second piston 6a is slid on the axis P1 along an inner wall of the housing 3 provided at a position of the inside of the second yoke 6c.

The adjustment valve 4 has a configuration in which the screw 4a, the coil spring 4b, and the plunger 4c are joined along the axis P1 and adjusts the fluid pressure in the stationary state of the first working fluid 41 in an increasing direction by increasing the pushing amount of the screw 4a.

The first artificial muscle unit 11 includes the first balloon 31 that is inflated and deflated by the fluid pressure of the first working fluid 41 and the first net 21 that covers the outer periphery of the first balloon 31. In addition, the second artificial muscle unit 12 includes the second balloon 32 that is inflated and deflated by the fluid pressure of the second working fluid 42 and the second net 22 that covers the outer periphery of the second balloon 32. Both the first net 21 and the second net 22 are plain weave and cylindrical. Both the first net 21 and the second net 22 are made of tough fibers such as carbon fibers. Both the first balloon 31 and the second balloon 32 are bags made of rubber such as silicone rubber. The first joining tube 8 and the second joining tube 9 are pressure-resistant tubes. Both the first working fluid 41 and the second working fluid 42 are working oils.

The first end portion 21a of the first artificial muscle unit 11 at a position close to the first joining tube 8 is joined to a position on the bending side of the second bone 52 at a position far from the joint 53 in the first bone 51. In addition, the second end portion 21b of the first artificial muscle unit 11 at a position far from the first joining tube 8 is joined to a position on the bending side of the second bone 52 at a position close to the joint 53 in the first bone 51. The third end portion 22a of the second artificial muscle unit 12 at a position close to the second joining tube 9 is joined to a position on the stretching side of the second bone 52 at a position far from the joint 53 of the first bone 51. In addition, the fourth end portion 22b of the second artificial muscle unit 12 at a position far from the second joining tube 9 is joined to a position on the stretching side of the second bone 52 at a position close to the joint 53 in the first bone 51.

The first working fluid 41 in the housing 3 is moved into the first balloon 31 via the first connection port 3a and the first joining tube 8 by the pushing force between the first piston 5a and the second piston 6a, and thus the first balloon 31 is inflated. Also, the first working fluid 41 in the first balloon 31 is moved into the housing 3 via the first joining tube 8 and the first connection port 3a by the attracting force between the first piston 5a and the second piston 6a, and thus the first balloon 31 is deflated. Then, the second working fluid 42 in the second balloon 32 is moved into the housing 3 via the second joining tube 9 and the second connection port 3b by the attracting force between the first piston 5a and the second piston 6a, and thus the second balloon 32 is deflated. Further, the second working fluid 42 in the housing 3 is moved into the second balloon 32 via the second connection port 3b and the second joining tube 9 by the pushing force between the first piston 5a and the second piston 6a, and thus the second balloon 32 is inflated. That is, the control circuit 17 drives and controls the actuator unit 2 by controlling the magnitude of the electric power applied to the actuator unit 2 and the direction of the energizing current.

In the above-described embodiment, an example in which the first artificial muscle unit 11 and the second artificial muscle unit 12 are combined to operate has been described. The present invention is not limited to this configuration, and three or more artificial muscle units can be combined to operate.

The present invention is not limited to the example described above, and various modifications can be made within the scope not departing from the present invention.

1. An artificial muscle actuator device comprising:
a first artificial muscle unit;
a second artificial muscle unit; and
an actuator unit, wherein
the artificial muscle actuator device is configured to be attached to a body part corresponding to a skeleton in which a first bone and a second bone are linked by a joint, and to perform a bending motion and a stretching motion with the first artificial muscle unit and the second artificial muscle unit,
the first artificial muscle unit includes
a first balloon that is inflated and deflated by fluid pressure of a first working fluid,
a first joining tube that joins the first balloon and the actuator unit to each other, and
a first net that covers an outer periphery of the first balloon and is attached to the body part,
the second artificial muscle unit includes
a second balloon that is deflated and inflated by fluid pressure of a second working fluid,
a second joining tube that joins the second balloon and the actuator unit to each other, and
a second net that covers an outer periphery of the second balloon and is attached to the body part, and
the actuator unit
includes a housing to which each of the first joining tube and the second joining tube is joined, a piston disposed in the housing, and a solenoid that moves the piston back and forth, and
is configured to perform a first motion of pushing the first working fluid toward the first balloon and drawing the second working fluid from the second balloon via a forward movement of the piston, and a second motion of pushing the second working fluid toward the second balloon and drawing the first working fluid from the first balloon via a backward movement of the piston.

2. An artificial muscle actuator device comprising:
a first artificial muscle unit;
a second artificial muscle unit; and
an actuator unit, wherein
the artificial muscle actuator device is configured to be attached to a body part corresponding to a skeleton in which a first bone and a second bone are linked by a joint, and to perform a bending motion and a stretching motion with the first artificial muscle unit and the second artificial muscle unit,
the first artificial muscle unit includes
a first balloon that is inflated and deflated by fluid pressure of a first working fluid,
a first joining tube that joins the first balloon and the actuator unit to each other, and
a first net that covers an outer periphery of the first balloon and is attached to the body part,
the second artificial muscle unit includes
a second balloon that is deflated and inflated by fluid pressure of a second working fluid,
a second joining tube that joins the second balloon and the actuator unit to each other, and
a second net that covers an outer periphery of the second balloon and is attached to the body part, and
the actuator unit
includes a housing to which each of the first joining tube and the second joining tube is joined, a first piston disposed in the housing, a first solenoid that moves the first piston back and forth, a second piston disposed in the housing, and a second solenoid that moves the second piston back and forth, and
is configured to perform a first motion of pushing the first working fluid toward the first balloon and drawing the second working fluid from the second balloon via a motion of separating the first piston and the second piston away from each other, and a second motion of pushing the second working fluid toward the second balloon and drawing the first working fluid from the first balloon via a motion of bringing the first piston and the second piston close to each other.

3. The artificial muscle actuator device according to claim 1, wherein
both the first balloon and the second balloon are made of oil-resistant elastic rubber,
both the first net and the second net are made of synthetic-fiber cylindrical fabric,
the cylindrical fabric has a plurality of thread-like fibers each being pulled out from both end portions, and
the thread-like fibers are moveable with the cylindrical fabric.

4. The artificial muscle actuator device according to claim 3, wherein
the first working fluid and the second working fluid are flame-retardant working oils,
the first balloon and the second balloon are made of silicone rubber, fluororubber, urethane rubber, or natural rubber, and
the first net and the second net are made of carbon fiber, polyester fiber, or polyamide fiber.

5. The artificial muscle actuator device according to claim 1, further comprising:
a first attachment tool that attaches, to the body part, a first connection portion of the first joining tube with a first end portion of the first net on a rear end side of the first net, and a second connection portion of the second joining tube with a third end portion of the second net on a rear end side of the second net; and
a second attachment tool that attaches, to the body part, a second end portion of the first net on a front end side of the first net and a fourth end portion of the second net on a front end side of the second net.

6. The artificial muscle actuator device according to claim 2, wherein
both the first balloon and the second balloon are made of oil-resistant elastic rubber,
both the first net and the second net are made of synthetic-fiber cylindrical fabric,
the cylindrical fabric has a plurality of thread-like fibers each being pulled out from both end portions, and
the thread-like fibers are moveable with the cylindrical fabric.

7. The artificial muscle actuator device according to claim 6, wherein
the first working fluid and the second working fluid are flame-retardant working oils,
the first balloon and the second balloon are made of silicone rubber, fluororubber, urethane rubber, or natural rubber, and
the first net and the second net are made of carbon fiber, polyester fiber, or polyamide fiber.

8. The artificial muscle actuator device according to claim 2, further comprising:
a first attachment tool that attaches, to the body part, a first connection portion of the first joining tube with a first end portion of the first net on a rear end side of the first net, and a second connection portion of the second joining tube with a third end portion of the second net on a rear end side of the second net; and
a second attachment tool that attaches, to the body part, a second end portion of the first net on a front end side of the first net and a fourth end portion of the second net on a front end side of the second net.
